(19) United States
(12) Patent Application Publication
(10) Pub. No.: US 2025/0261905 A1
(43) Pub. Date: Aug. 21, 2025

(54) HORMONAL HEALTH COACHING BASED ON CARDIAC AMPLITUDE

(71) Applicant: Whoop, Inc., Boston, MA (US)

(72) Inventors: Emily Rachel Capodilupo, Boston, MA (US); Summer Rose Jasinski, Cambridge, MA (US); David Mikal Presby, Bern (CH); Victoria Harrison Lee, Waltham, MA (US)

(21) Appl. No.: 19/058,997

(22) Filed: Feb. 20, 2025

Related U.S. Application Data
(60) Provisional application No. 63/555,753, filed on Feb. 20, 2024.
(60) Provisional application No. 63/561,411, filed on Mar. 5, 2024.

(51) Int. Cl. A61B 5/00; A61B 5/0205; A61B 5/024; A61B 10/00

(52) U.S. Cl. CPC A61B 5/486; A61B 5/0205; A61B 5/02405; A61B 5/02416; A61B 5/7275; A61B 10/0012; A61B 2010/0019; A61B 2010/0029

(57) ABSTRACT

A model for a cardiovascular amplitude metric characterizes timewise changes in a cardiac metric for a user based on a follicular mean of the cardiac metric and a luteal mean of the cardiac metric. The cardiovascular amplitude metric can be calculated for the user, e.g., based on data from a wearable physiological monitor, and used to provide coaching for fertility, hormonal health, fitness, and so forth.

### RELATED APPLICATIONS

This application claims priority to U.S. Prov. App. No. 63/555,753 filed on Feb. 20, 2024, and U.S. Prov. App. No. 63/561,411 filed on Mar. 5, 2024. The entire content of each of the foregoing applications is hereby incorporated by reference.

### TECHNICAL FIELD

The present disclosure generally relates to tracking hormonal cycles based on physiological monitoring, and more specifically providing related recommendations and coaching for reproductive phases, pregnancy, menopause, fertility, and the like.

### BACKGROUND

Reproductive phases may affect health, fitness, recovery, sleep, and the like. There remains a need for techniques to characterize reproductive phases in a manner that facilitates coordinated delivery of coaching and recommendations related to aspects of hormonal health such as pregnancy, menopause, fertility, and the like.

### SUMMARY

A model for a cardiovascular amplitude metric characterizes timewise changes in a cardiac metric for a user based on an offset between a follicular mean of the cardiac metric and a luteal mean of the cardiac metric. The cardiovascular amplitude metric can be calculated for the user, e.g., based on data from a wearable physiological monitor, and used to provide coaching for fertility, hormonal health, fitness, and so forth.

In one aspect, a computer program product disclosed herein includes computer executable code embodied in a non-transitory computer readable medium that, when executing on one or more computing devices, causes the one or more computing devices to perform the steps of: providing a model for a cardiovascular amplitude metric that characterizes timewise changes in a cardiac metric of a user during a model hormonal cycle based on an offset between (a) a follicular mean over a first interval of at least three sequential daily measurements of resting heart rate or heart rate variability that includes day five of the model hormonal cycle and (b) a luteal mean over a second interval of at least three sequential daily measurements of a resting heart rate or a heart rate variability that includes day twenty five of the model hormonal cycle; acquiring heart rate data for the user from a wearable photoplethysmography monitor during a user hormonal cycle; calculating the cardiovascular amplitude metric of the user for the user hormonal cycle with the model for the cardiovascular amplitude metric; and providing feedback to the user based on the cardiovascular amplitude metric.

Providing feedback to the user may include displaying the cardiovascular amplitude metric to the user. Providing feedback to the user may include: estimating a phase of the user in a current hormonal cycle; determining a confidence level for the phase based on the cardiovascular amplitude metric; and reporting the confidence level for the phase to the user. Providing feedback to the user may include providing fertility coaching. Providing fertility coaching may include: estimating a phase of the user in a current hormonal cycle; calculating an ovulation time of the user based on the phase; and reporting a confidence level in the ovulation time to the user based on the cardiovascular amplitude metric. Providing fertility coaching may include reporting a likelihood of conception to the user. Providing fertility coaching may include providing suggestions for one or more behavioral modifications to increase the cardiovascular amplitude metric. Providing fertility coaching may include: determining a phase of the user based on data from the wearable photoplethysmography monitor; storing one or more ovulation observations by the user in a memory; predicting an ovulation time for the user based on a combination of the phase of the user an the one or more ovulation observations; and providing a fertility coaching recommendation to the user including the ovulation time, a likelihood to conceive based on the cardiovascular amplitude metric, and an accuracy of the ovulation time based on the cardiovascular amplitude metric. Providing feedback may include providing an indicator to the user of a possible onset of perimenopause or menopause.

In another aspect, a method disclosed herein includes: providing a model for a cardiovascular amplitude metric that characterizes timewise changes in a cardiac metric for a user based on an offset between a follicular mean of the cardiac metric and a luteal mean of the cardiac metric; acquiring physiological data for the user from a wearable monitor during a hormonal cycle, where the physiological data may include heart rate data for the user; calculating the cardiovascular amplitude metric of the user with the model for the hormonal cycle based on the physiological data; and providing feedback to the user based on the cardiovascular amplitude metric.

Providing feedback to the user may include displaying the cardiovascular amplitude metric to the user. Providing feedback to the user may include providing fertility coaching. Providing fertility coaching may include: estimating a phase of the user in a current hormonal cycle; calculating an ovulation time of the user based on the phase; and reporting a confidence level in the ovulation time to the user based on the cardiovascular amplitude metric. Providing fertility coaching may include reporting a likelihood of conception to the user. Providing fertility coaching may include: determining a phase of the user based on data from the wearable monitor; storing one or more ovulation observations by the user in a memory; predicting an ovulation time for a user based on a combination of the phase of the user and the one or more ovulation observations; and providing a fertility coaching recommendation to the user including the ovulation time, a likelihood to conceive based on the cardiovascular amplitude metric, and an accuracy of the ovulation time based on the cardiovascular amplitude metric.

The follicular mean may include a first mean of a first sequence of cardiac measurements over a first interval around an expected follicular turning point of the cardiac metric for the user, and the luteal mean may include a second mean of a second sequence of cardiac measurements over a second interval around an expected luteal turning point of the cardiac metric for the user. In one aspect, the cardiac metric may include a resting heart rate; the expected follicular turning point of the cardiac metric occurs on day five of the hormonal cycle; the expected follicular turning point is a minimum of the resting heart rate; the first interval is at least seven days; the expected luteal turning point of the cardiac metric occurs on day twenty five of the hormonal cycle; the expected luteal turning point is a maximum of the resting heart rate; and the second interval is at least seven days. In another aspect, the cardiac metric may include a heart rate variability; the expected follicular turning point of the cardiac metric occurs on day five of the hormonal cycle; the expected follicular turning point is a maximum of the heart rate variability; and the first interval is at least three days. In another aspect, the cardiac metric may include a heart rate variability; the expected luteal turning point of the cardiac metric occurs on day twenty five of the hormonal cycle; the expected luteal turning point is a minimum of the heart rate variability; and the second interval is at least three days.

In another aspect, a system disclosed herein includes a wearable physiological monitor configured to acquire cardiac data from a user; a memory storing a model for a cardiovascular amplitude metric that characterizes timewise changes in a cardiac metric of a user during a model hormonal cycle, where the model is based on an offset between a first mean of a first interval of measurements around an expected follicular turning point of the cardiac metric for the user and a second mean of a second interval of measurements around an expected luteal turning point of the cardiac metric for the user; and a processor configured by computer executable code to receive data from the wearable physiological monitor over a hormonal cycle for the user, calculate the cardiovascular amplitude metric for the hormonal cycle, and generate feedback to the user based on the cardiovascular amplitude metric.

In another aspect, a method disclosed herein includes providing a model for a physiological signal amplitude metric that characterizes timewise changes in a physiological metric for a user based on an offset between a follicular mean of the physiological metric and a luteal mean of the physiological metric; acquiring physiological data for the user during a hormonal cycle, where the physiological data includes data relating to the physiological metric for the user; calculating the physiological signal amplitude metric of the user with the model for the hormonal cycle based on the physiological data; and providing feedback to the user based on physiological signal amplitude metric.

The physiological metric may include a skin temperature of the user. The physiological metric may include a respiratory rate. The physiological metric may include at least one of a resting heart rate and a heart rate variability for the user. Acquiring physiological data may include acquiring the physiological data from a wearable monitor worn by the user during the hormonal cycle.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a method to calculate a cardiovascular amplitude metric for a user.

FIG. 7 illustrates a model for measuring cardiovascular amplitude.

FIG. 8 shows model statistics for RHR and HRV model for naturally cycling individuals.

FIG. 9 shows results of GLM for RHRₐₘₚ and HRVₐₘₚ.

FIG. 10 shows GLM model results.

FIG. 11 shows cardiovascular amplitude and metric offset values for control and birth control cohort.

FIG. 12 shows a comparative distribution of a cardiovascular amplitude metric for participants using a birth control pill and naturally cycling participants.

FIG. 13 shows a method for calculating and using a cardiovascular amplitude metric.

### DESCRIPTION

Embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night), etc. More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example increasing the sampling rate when signal variability increases, or during periods of relatively higher motion, or based on user input.

At the same time, continuous monitoring is not intended to exclude ordinary data acquisition interruptions such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data such as optical intensity signals, or processed data therefrom such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component or module of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as, DRAM, SRAM, EDO RAM), and so forth. Although not depicted, any of the devices or components described herein may include a computer-readable medium or other memory for storing program instructions, data, and the like.

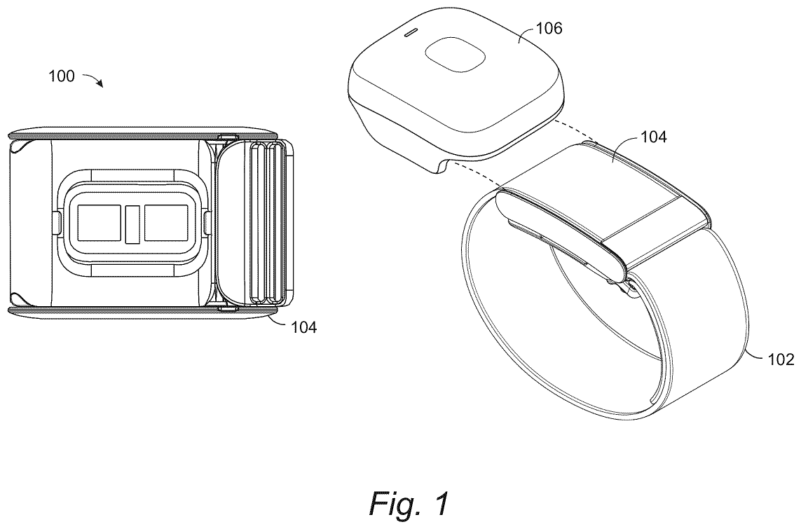

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104 and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiography other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure (via physical pressure measurements or other means), sound, electrocardiograms, and the like, as well environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting addition functions of the wearable monitor 104. For example, the system 100 may include communications systems to support, e.g., near field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a GeoPositioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

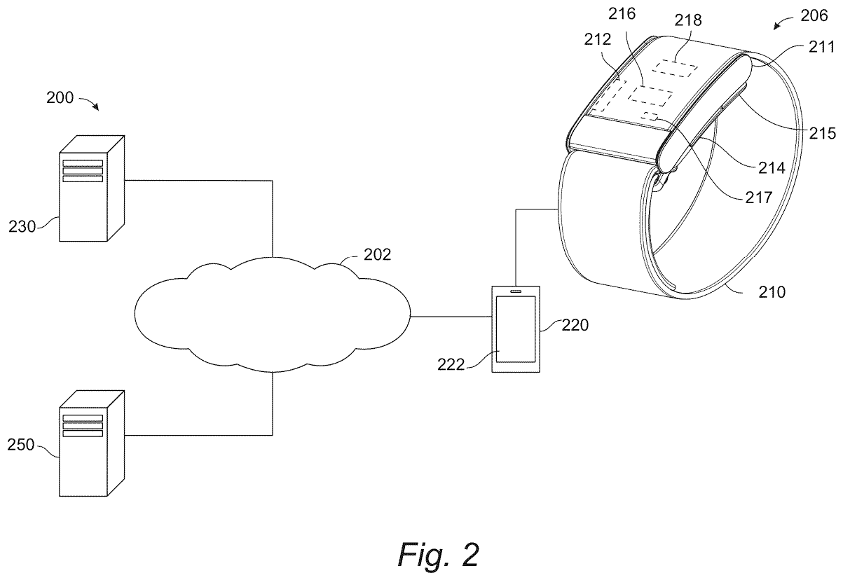

FIG. 2 illustrates a physiological monitoring system. More specifically, FIG. 2 illustrates a system 200 for physiological monitoring that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communicating with local resources. By way of non-limiting examples, short range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211 or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

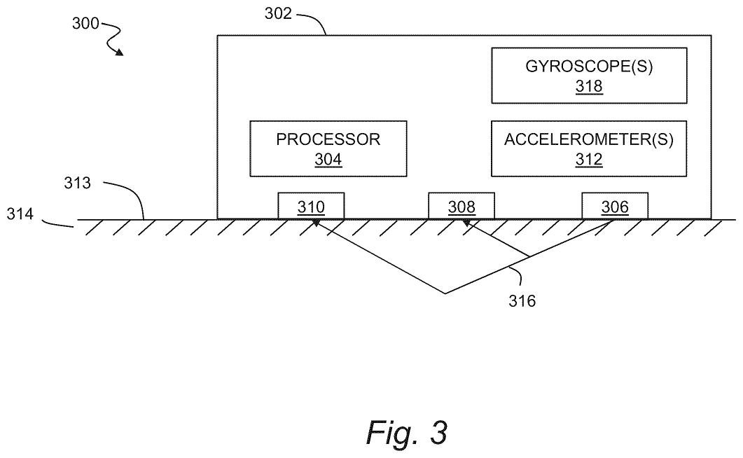

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like such as audio earbuds, earrings or similar, glasses and/or other eyewear, a ring, a headband, and so forth.

The processor 304 may be any microprocessor, microcontroller, application specific integrated circuit, or other processing circuitry or combination of the foregoing suitable for controlling operation of the physiological monitor and acquiring physiological data.

The light source 306 may include one or more light emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light emitting diodes that emit light in the green, red, infrared, near infrared, or other suitable wavelength ranges, which can provide desired light transmission through human skin, facilitating low power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308, 310 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

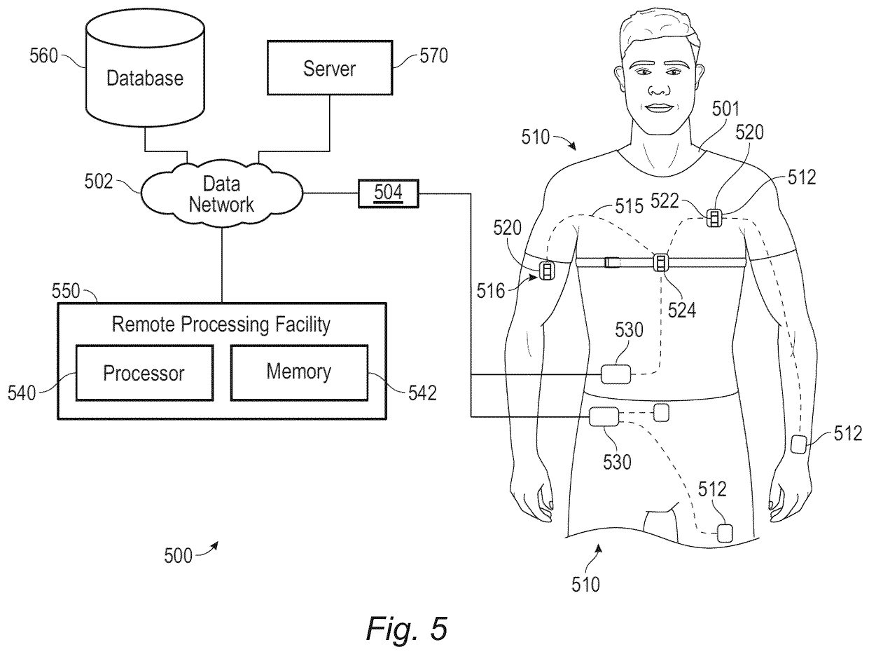

The physiological monitors described herein—e.g., in the systems described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

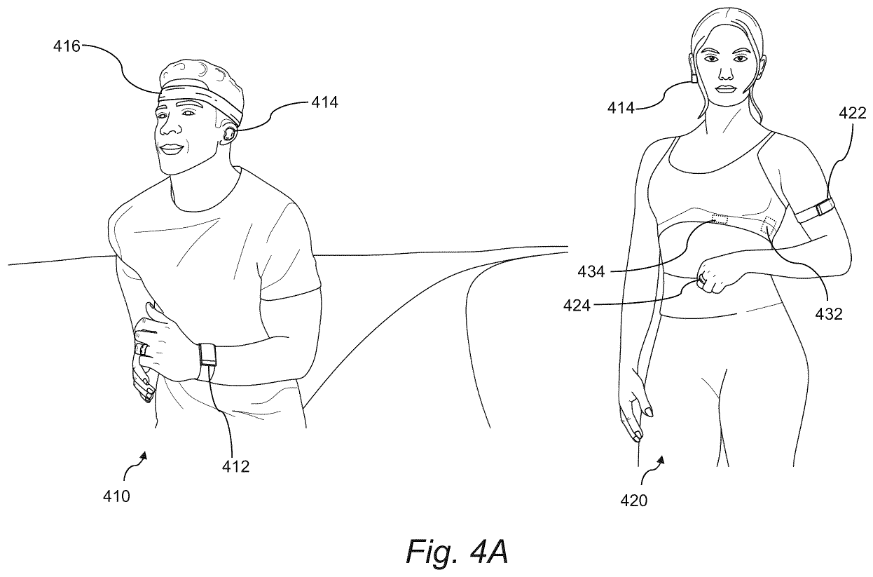

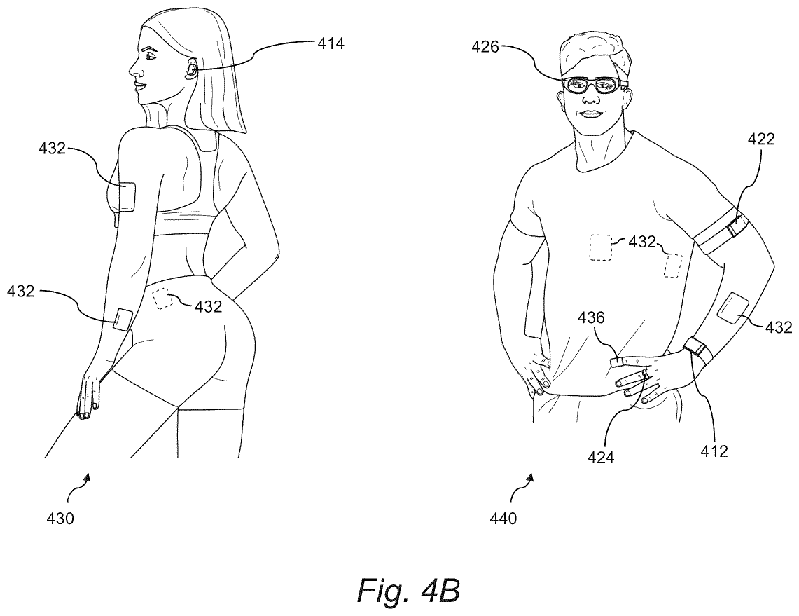

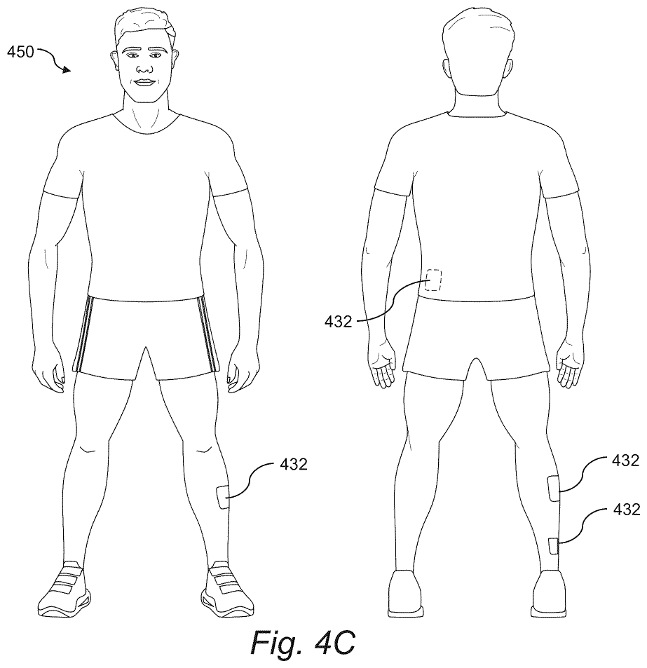

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors such as a wrist-worn device 412 (such as any described herein), an ear-worn device 414 (including on-ear devices retained with a clamp, clip, or other mechanism, and/or in-ear devices such as earbuds or the like that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large scale motion such as geographical movement and elevation changes that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors, and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to a pulse oximetry or SpO₂) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smart phone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some aspects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smart phone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as wi-fi or cellular signals, and converting them into usable electrical power), ambient light harvesting, and so forth. Other techniques may also or instead be used to provide external power, such as beam steering or resonant techniques for short range or medium range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system to be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smart phone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interactions, for example by storing data locally and transmitting the data over available local area network resources when a local access point such as a wireless access point or a relay device (such as a laptop, tablet, or smart phone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for received light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 524 may be any as described herein, for example including any of the features of the network interface 504 described above.

The controller 530 may be configured, e.g., by computer executable code or the like, to determine a location of the module 520. This may be based on contextual measurements such as accelerometer data from the module 520, which may be analyzed by a machine learning model or the like to infer a body position. In another aspect, this may be based on other signals from the module 520. For example, signals from sensors such as photodiodes, temperature sensors, resistors, capacitors, and the like may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500 such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes, and may, for example, be used to improve the design or use of modules 520 and garments 510, either for a population, for a user type, or for a particular user.

As stated above, the system 500 may further include a processor 540 and a memory 542. In general, the memory 542 may bear computer executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like as shown in the figure. Thus, in an aspect, one or more of the processor 540 and the memory 542 is included on at least one of the plurality of modules 520. In this manner, processing may be performed on a central module, or on each module 520 independently. In another aspect, one or more of the processor 540 and the memory 542 is remote relative to each of the plurality of modules 520. For example, processing may be performed on a connected peripheral device such as smart phone, laptop, local computer, or cloud resource.

The processor 540 may be configured to assess the quality of the data received from a physiological sensor 522 of the module 520, otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources, may advantageously be used in combination to improve physiological monitoring and achieve modes of monitoring not previously available.

Coaching based on cardiovascular amplitude.

Disruptions to the menstrual cycle outside of pregnancy may indicate an underlying health concern. For instance, missed periods may often be related to low estrogen and low progesterone, with the latter resulting in health complications such as premenstrual dysphoric disorder. An irregular menstrual cycle may also indicate or cause an increased risk of other health complications such as coronary heart disease, cancer, and osteoporosis. As described herein, wearable devices and technology can provide continuous and accurate measures of physiological data such as skin temperature, heart rate, resting heart rate (RHR), heart rate variability (HRV), respiration rate, and so forth. These metrics can facilitate automated detection and tracking of hormonal cycles, as well as detection of irregularities. Wearable devices may also be used for automatic tracking of fertility, pregnancy status, menopause, and other conditions linked to the hormonal cycle. This type of tracking may be supplemented by other relevant information such as age, body mass index (BMI), height, weight, hormone therapy or other medication usage, and/or birth control usage in order to improve monitoring of the menstrual cycle and related physiological data, and to provide insights to assist a user in managing hormonal medications, therapies, and trends. As described herein, hormonal cycle management is intended to include providing coaching or other recommendations to a user based on short term (e.g., a single menstrual cycle) or long term (e.g., postmenarcheal to menopausal) hormonal phases, or to provide coaching or other recommendations for managing the hormonal cycle itself, e.g., with targeted therapies, lifestyle adjustments, and so forth.

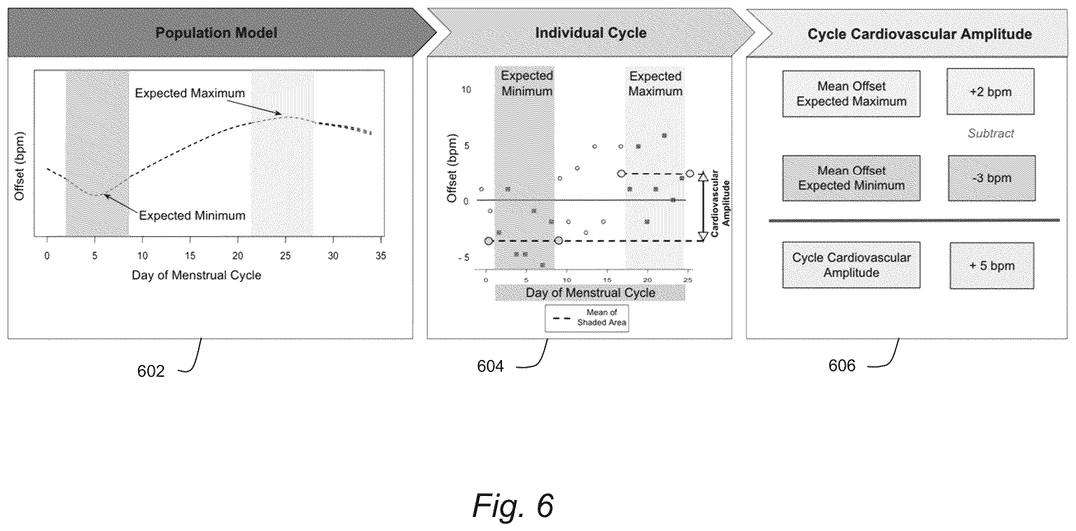

FIG. 6 shows a method to calculate a cardiovascular amplitude metric for a user. In general, a menstrual cycle may be defined as the time between onsets of menstrual bleeding. In order to track this cycle using data from a wearable monitor, RHR and HRV may be normalized for a user within cycles by calculating the offset in absolute units from the cycle mean value. For a given data set, outliers may usefully be removed (e.g., as potentially erroneous) when calculating normalized RHR/HRV values for a cycle. For example, outliers may be defined as values above quartile three (Q3)+1.5×IQR (the interquartile range) or below quartile one (Q1)−1.5×IQR for a given user and menstrual cycle, or using any other suitable filter or combination of filters for removing questionable data from the analysis.

The partial dependence plots 602 for a population model illustrate the days within a standard menstrual cycle in which an individual's RHR or HRV may be expected to reach a maximum or minimum value. The expected maximum value and expected minimum value may be determined by the nearest day to the maximum and minimum of the partial dependence plot in a population-level Generalized Additive Mixed Model (GAMM), or any other GAM or other suitable model. A cycle amplitude for each participant cycle may be based on the individual cycle data 604, and may be calculated, e.g., as the difference 606 between the seven-day mean centered on the expected peak and the seven-day mean centered on the expected nadir. For example, if a population model suggested a maximum on day four and a minimum on day fourteen, the cycle cardiovascular amplitude value for each participant-cycle may be determined by subtracting the mean of days eleven through seventeen from the mean of days one through seven. The mean of the cycle amplitudes from all of the participant's eligible menstrual cycles may be calculated in order to establish intervals for an individual cardiovascular amplitude metric. It will be understood that, while a particular modeling technique is described herein for characterizing the daily amplitude of a physiological metric such as resting heart rate or heart rate variability, a variety of other techniques may also or instead be used to characterize daily variations over a menstrual cycle based on population-wide or single-user historical data. Furthermore, a variety of other physiological metrics may also or instead be used, such as respiration rate, body temperature, and so forth. Thus, in one aspect, the techniques described herein may be used with a physiological signal amplitude metric based on a physiological metric such as skin temperature, respiratory rate, and the like, any of which may have an intracycle amplitude change indicative of hormonal health.

A seven-day interval may be used, based on a mean of sequential daily values for the corresponding cardiac metric, in order to mitigate the impact of day-of-week effects, where, for example, weekend activity and corresponding physiological measurements may vary significantly from weekday activities. However, other windows may also or instead be used, such as at least three days, five days, or some other interval. It will further be appreciated that the measurement window need not be centered on the expected turning point (e.g., the nadir or peak) for the cardiac metric, and may be offset chronologically, e.g., to avoid sampling data from adjacent menstrual cycles or to weight the sampling according to expected asymmetries in the time varying data. For example, if a maximum or minimum is fewer than three days from the beginning or end of a cycle, the seven-day period may be shifted to increase the amount of data available for a participant. The amount of data may be increased for reasons such as but not limited to a determination being made that there is not sufficient data available for each subject. As used herein, the derived participant cardiovascular amplitude values are denoted as HRVₐₘₚ and RHRₐₘₚ for HRV cardiovascular amplitude and RHR cardiovascular amplitude metrics, respectively.

In one aspect, generalized linear models (GLM) may be used to evaluate the relationship between known mediators of hormonal health and the cardiovascular amplitude metric as described herein. For example, a GLM may be fit with the participant's age (age), resting heart rate (RHR), and BMI (BMI) as covariates to estimate values of HRVₐₘₚ. Other data may also or instead be used, such as baseline activity for a user or birth control status. Natural spline functions with four knots may be selected to model RHR data, while age and BMI may be modeled linearly. This permits different characteristic curves within the cycle to be represented by different splines—e.g., piecewise polynomial functions—which can be connected in turn at the knots with equal values so that the splines remain continuous across each knot, and across the domain of the splines. A natural spline adds extra constraints to ensure that the function is not only smooth, but has a controlled behavior at the ends. For example, a cubic natural spline requires that the second derivative of the spline function is zero at the endpoints of the domain so that the spline remains linear at (and beyond) the boundary knots.

The general formula of the GLM for RHRₐₘₚ may be expressed as:

$${RHR}_{amp} = {β}_{0} + {β}_{1} * age + {β}_{2} * ns ( RHR , 4 ) + {β}_{3} * BMI + ε$$

The general formula of the GLM for HRVₐₘₚ may be expressed as:

$${HRV}_{amp} = {β}_{0} + {β}_{1} * age + {β}_{2} * ns ( HRV , 4 ) + {β}_{3} * BMI + ε$$

In one aspect, cardiovascular amplitude metrics may be calculated for a number of participants who use the birth control pills or other medication that may affect hormones or the menstrual cycle, e.g. using the days for the expected minima and maxima derived from the naturally cycling population model. The cardiovascular amplitudes of the naturally cycling participants may then be compared to the cardiovascular amplitudes of the participants using birth control pills or other medication, and the two different numbers for each group of participants may be matched on baseline metrics such as RHR or age to create comparable populations. Various techniques and tools exist to match treatment and control groups, and may be used to create comparable populations of treated and untreated groups for purposes of modeling RHR and HRV during hormonal cycles.

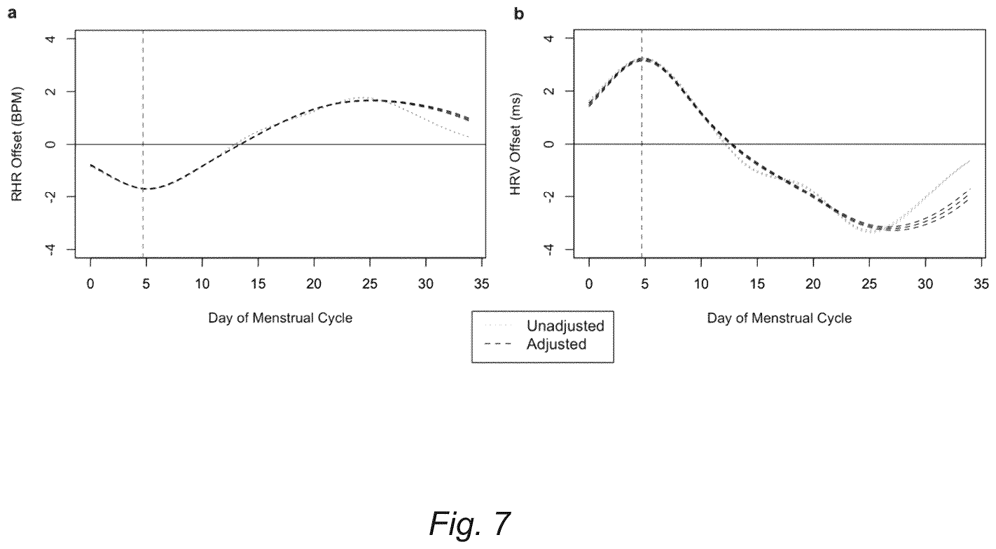

FIG. 7 illustrates a model for measuring cardiovascular amplitude. More specifically, FIG. 7 shows results of a population-level GAM of HRV offset and RHR offset during the menstrual cycle, including population-level minima and maxima for RHR and HRV. The adjusted and unadjusted models for RHR offset values are displayed in (a), and adjusted and unadjusted models for HRV offset are displayed in (b). A vertical dashed line indicates the typical end of menstrual bleeding and provides a useful benchmark for measuring menstrual cycles. Boundary lines indicate the 95% confidence interval for estimates.

A population-level GAM of naturally cycling participants may be used to confirm or determine an association between a particular instance of time in a menstrual cycle (e.g., a particular day) and cardiovascular metrics, and further to determine a pattern or function of how RHR offset and HRV offset may change throughout the menstrual cycle. This GAM may be used to determine the HRV offset or RHR offset as a function of the day of a menstrual cycle. The GAM may be adjusted and recreated using different variables resulting in a plurality of models that may be used to determine the HRV offset or RHR offset as a function of the day of the menstrual cycle. A GAM may be a population-level model, sub-group-level model, or individual model. For example, a GAM may be selected or created with variables to provide a model that applies to an individual or group based on characteristics associated with that individual or group. One or more of these functions may be used with RHR offset or HRV offset values for an individual to evaluate the phase (e.g., the day relative to a predetermined starting point of the cycle) of an individual's menstrual cycle, or to calculate other information related to an individual's menstrual cycle such as irregularities. The maximum or minimum of the HRV offset or RHR offset as a function of a day in a menstrual cycle for data in a population model may be used to determine a day of a menstrual cycle for a user, or to determine other menstrual cycle information for the user such as irregularities. The value of the HRV offset may be used to determine or estimate the RHR offset or the rate of change of the RHR offset in the same menstrual cycle. The offset of the RHR may also or instead be used to calculate an HRV offset or the rate of change of the HRV offset in the same menstrual cycle, and the rate of change of the HRV offset or RHR offset as a function of time in a GAM may be used to determine a day of the menstrual cycle for the user, or to determine other information related to a menstrual cycle such as irregularities. A GAM may model other physiological or cardiovascular metrics other than an HRV offset or RHR offset, any of which may be used—if suitably correlated to the menstrual cycle—to evaluate where a user is in the menstrual cycle, and to provide a basis for calculating an offset for cardiovascular amplitude as described herein.

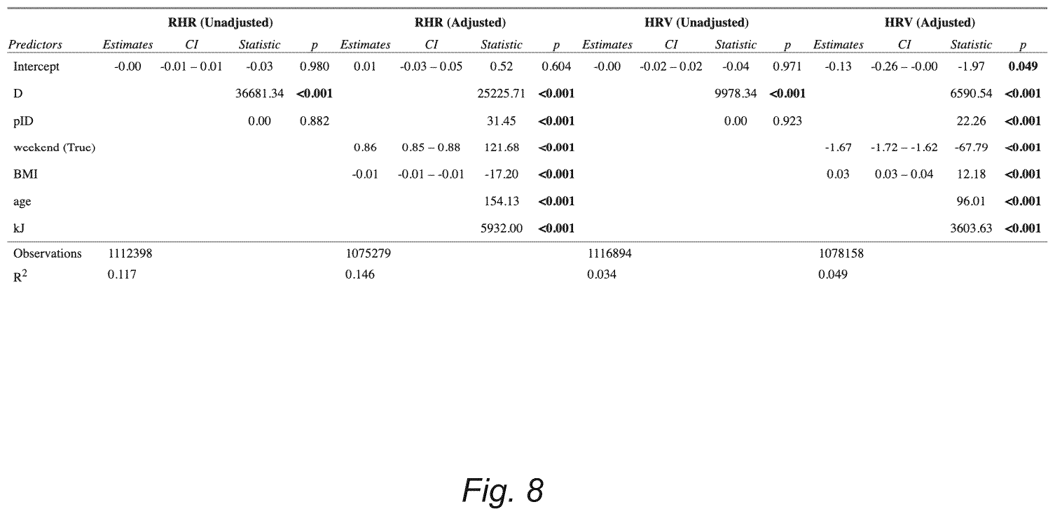

FIG. 8 shows model statistics for RHR and HRV in a model for naturally cycling individuals. The statistics include estimates, confidence intervals (CI), test statistics, and p-values for the predictors in the adjusted (n=1,112,398) and unadjusted (n=1,075,279) population GAM models of RHR offset as well as the adjusted (n=1,116,894) and unadjusted (n=1,078,158) population GAM models of HRV offset. For the RHR (Unadjusted) and RHR (Adjusted) output, the model illustrates a relationship between D, pID, weekend, BMI, age, and kJ on the offset of RHR throughout a menstrual cycle in naturally cycling individuals. For the HRV (Unadjusted) and HRV (Adjusted) output, the model illustrates the relationship between D, pID, weekend, BMI, age, and kJ on the offset of HRV throughout the menstrual cycle in naturally cycling individuals.

In this context, the mean cycle amplitude for RHR may be calculated as a difference between a first mean value (for a first range of days around a first selected day, such as expected turning point in RHR) and a second mean value (for a second range of days around a second selected day, such as a second turning point in RHR). Here, RHRₐₘₚ may be calculated as each participant's mean per-cycle cardiovascular amplitude across all eligible menstrual cycles. The first day may be the maximum expected point in the population model for the RHR and the second day may be the minimum expected point in the population model for the RHR. In response to variable lengths of menstrual cycles, instead of centering the second mean value on the day where the minimum value occurred, the second mean value may be calculated using a range of days substituted for equivalent data in each cycle. In some cases, the range of days may be a range which ends on the last day of a menstrual cycle, or a range centered on, e.g., day twenty-five of the menstrual cycle. Therefore, a cardiovascular amplitude for RHR in this context may be defined as a first mean of measurements around an expected maximum (in the luteal phase) minus a second mean of measurements around an expected minimum (in the follicular phase), averaged across a number of available cycles.

In one example, a first seven-day mean value may be centered about a maximum expected point occurring on approximately day five in a population model for cardiovascular amplitude and a second seven-day mean value may be centered about a minimum expected point on approximately day twenty-six. Because menstrual cycles typically range from twenty-one to thirty-five days, instead of centering the second seven-day mean value on the day where the minimum value occurred (day twenty-six), the mean value of the seven finals days may be substituted to equivalent data in each cycle when calculating mean values for a user. In response to variable lengths of menstrual cycles, instead of centering the second mean value on the day where the minimum value occurred, the second mean value may be calculated using a range of days substituted for equivalent data in each cycle.

In this context, the mean cycle amplitude for HRV may be calculated as a difference between a first mean value (for a first range of days centered about a first day, such as a first turning point in the HRV) and a second mean value (for a second range of days centered about a second selected day, such as a second turning point in the HRV). The cardiovascular amplitude metric, RHRₐₘₚ, may be calculated as each participant's mean per-cycle cardiovascular amplitude across all eligible menstrual cycles. The first day may be the minimum expected point in the population model for the HRV and the second day may be the maximum expected point in the population model for the HRV. Because menstrual cycles vary in length, instead of centering the first mean value on the day where the minimum value occurred, the first mean value may be calculated using a third range of days substituted for equivalent data in each cycle. In some cases, the third range of days may be a range which ends on the last day of a menstrual cycle.

In the empirical data summarized in FIG. 8, a population level GLM suggest a negative association between age and RHRₐₘₚ, as well as age and HRVₐₘₚ. While the baseline value of a given metric may be a significant factor in the final cardiovascular amplitude, the relationship may not be linear. For example, one model suggests that RHRₐₘₚ estimates peak when baseline values are at the 50^{th}-75^{th} percentile while HRVₐₘₚ peaks when baseline values are at the 25^{th}-50^{th} percentile. BMI in this analysis was not significantly associated with HRVₐₘₚ but there may be a statistically significant association between BMI and RHRₐₘₚ.

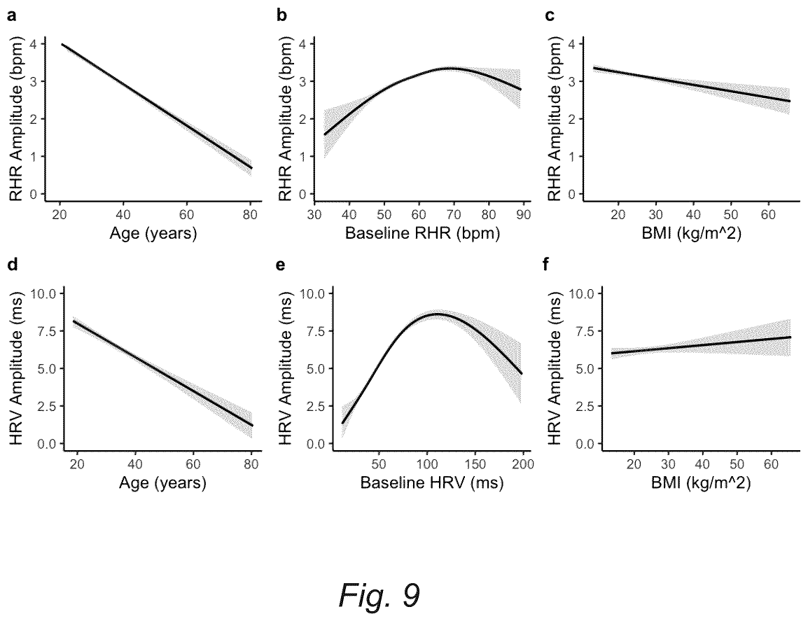

FIG. 9 illustrates a Generalized Linear Model (GLM) for RHRₐₘₚ and HRVₐₘₚ, showing the relationship between selected, known mediators of hormonal health and the cardiovascular amplitude metrics described herein. More specifically, FIG. 9 illustrates the partial dependence of age for (a) RHRₐₘₚ and (d) HRVₐₘₚ. The partial dependence of the four spline terms of baseline RHR is plotted for RHRₐₘₚ (b) and the partial dependence of the four spline terms of baseline HRV is plotted for HRVₐₘₚ (e). The partial dependence of BMI is also plotted for (c) RHRₐₘₚ and (e) HRVₐₘₚ. Boundary lines indicate the 95% confidence interval for estimations.

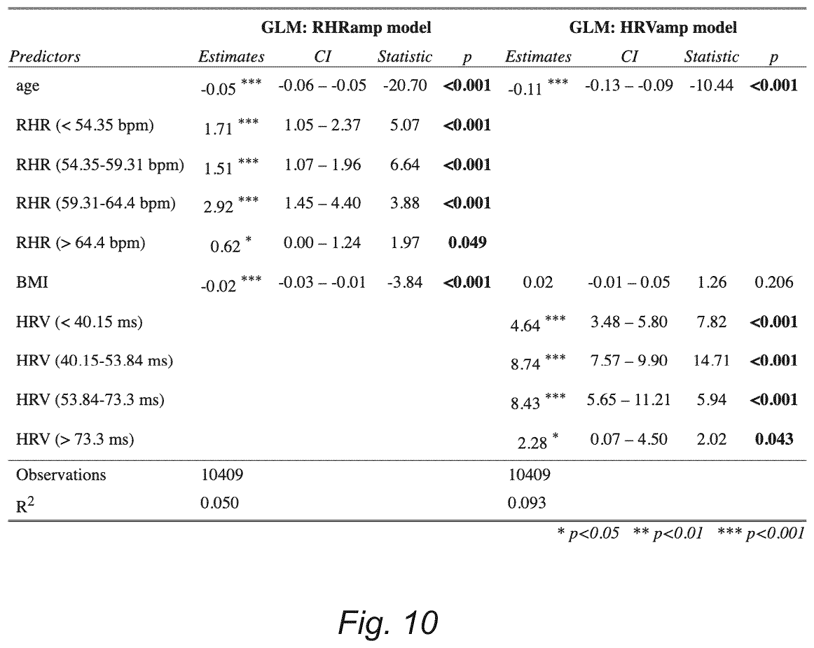

FIG. 10 shows GLM results. More specifically, the table in FIG. 10 illustrates the relationship between D, pID, weekend, BMI, age, and kJ on the offset of RHR throughout the menstrual cycle in naturally cycling individuals for one studied group of about 10,000 individuals.

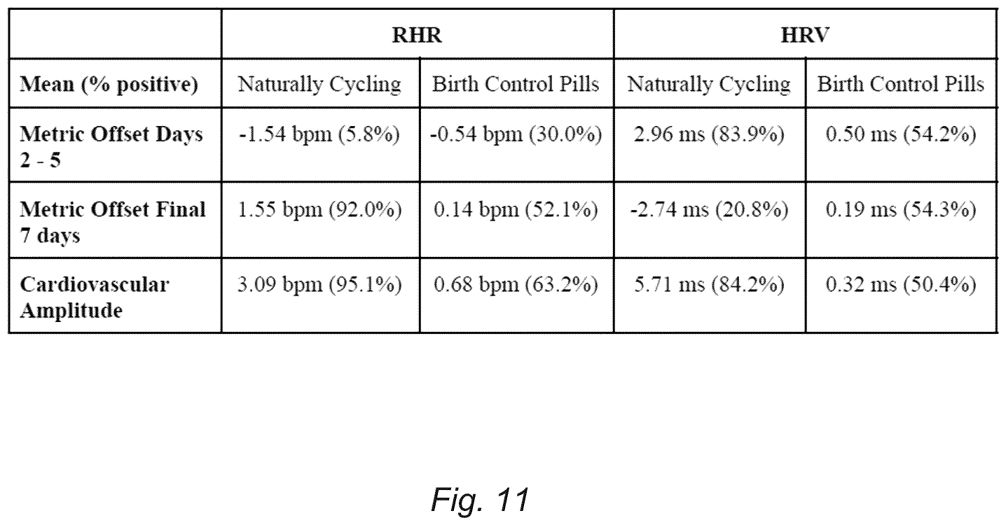

FIG. 11 shows cardiovascular amplitude and metric offset values for naturally cycling (control) and birth control (experimental) cohorts in one study. The table provides mean values (% of the population with a positive value) for the offset of the underlying metric of RHR and HRV for days 2-5 of the menstrual cycle, the final seven days of the menstrual cycle, and the difference between the two (cardiovascular amplitude) for both the control and experimental cohorts. For RHRₐₘₚ, the mean value was 3.09 bpm (±1.99) for the naturally cycling cohort, and 0.69 bpm (±2.03) for participants using birth control pills. For HRVₐₘₚ, the mean value was 5.71 ms (±7.36) for those naturally cycling and 0.31 ms (±7.05) for those using birth control pills. Other studies have yielded similar results.

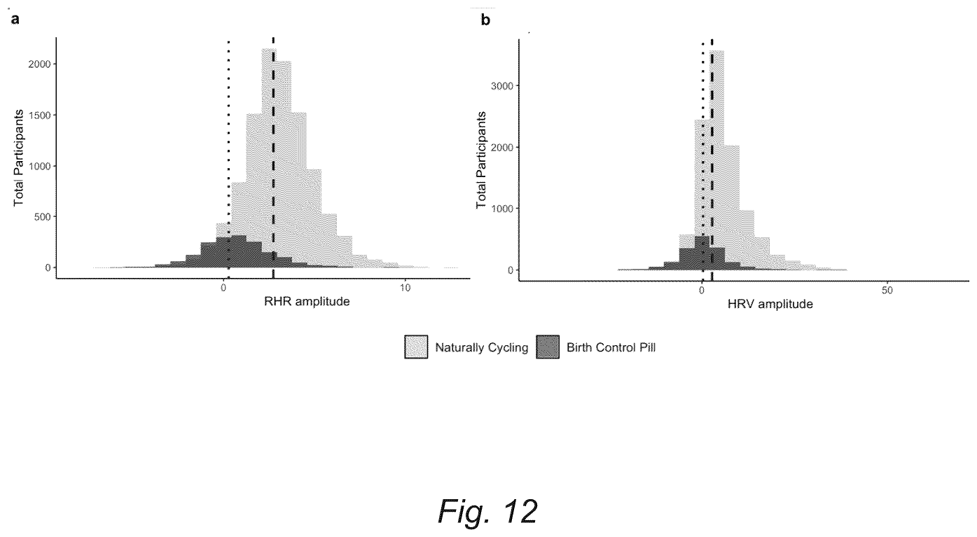

FIG. 12 shows a comparative distribution of a cardiovascular amplitude metric for participants using a birth control pill and naturally cycling participants.

The figures and analysis discussed herein show that for the studied population, a cardiovascular amplitude as described herein fluctuates in a regular and predictable pattern across the menstrual cycle, and that fluctuations for RHR and HRV are moderated with greater age, increased BMI, or the use of hormonal birth control. The RHR and HRV fluctuate substantially and with consistent patterns across the menstrual cycle both at the population and individual levels. For naturally cycling and regularly menstruating individuals, the lowest RHR and the highest HRV values occur at the end of menstruation (i.e. days three through seven) while the highest RHR and lowest HRV values occur in the late luteal phase (i.e. days twenty-five through twenty-seven). This pattern of menstrual cycle-related fluctuation in RHR and HRV metrics can be explained by cycle-related fluctuations in the reproductive hormones associated with the hormonal cycle and provide a reliable indicator of female reproductive hormone variations across the menstrual cycle. As such, the cardiovascular amplitude metric described herein provides a useful basis for quantitatively monitoring hormonal states and providing related feedback to the user. A number of applications of this cardiovascular amplitude metric to feedback and coaching are now described.

In one aspect, reduced cardiovascular amplitude is negatively associated with age and body mass index. This reduction in cardiovascular amplitude in older females may be affected by the gradual decline in anti-müllerian hormone, progesterone, and estrogen (i.e., reproductive hormones) among females throughout their reproductive life and approaching their final menstrual period, and provides a basis for detection and coaching in a pre-menopausal or menopausal context.

In another aspect, individuals with abnormal BMI (at either extreme) may be more likely to have complications such as but not limited to anovulatory cycles, irregular menses, hormonal imbalances, and other irregularities. Premenopausal females with an elevated BMI may exhibit reduced levels of estrogen and anti-müllerian hormone. Attenuated HRVₐₘₚ and RHRₐₘₚ in participants with abnormal BMI appear related to these underlying differences in hormonal health. As such, the cardiovascular amplitude metric described herein may also or instead provide a basis for behavioral coaching related to BMI.

In one aspect, cardiovascular amplitude may be aggregated over two or more cycles to determine an “average amplitude” metric and “variation in amplitude” metric. Increases or decreases in this metric over time may provide an indicator of, e.g., menopausal onset, fertility, hormone levels, and so forth. Thus, cycle regularity may be evaluated using a cardiovascular amplitude metric, which can provide a basis for corresponding inferences about hormonal levels and cycles to support coaching and behavioral recommendations. While RHR and HRV are described here in detail, other suitable physiological metrics include but are not limited to age, BMI, blood pressure, respiratory rate, body temperature, oxygen saturation, blood glucose level, muscle mass, muscle strength, cholesterol levels, cerebral blood flow, urine output, peak expiratory flow rate, pH level of bodily fluids, hormone levels, blood oxygen tension, and so forth.

According to the foregoing, a cardiovascular amplitude metric may be used to quantitatively evaluate the hormonal cycle of an individual and may be used to support coaching, recommendations, and management related to hormone levels, reproductive cycles, fertility, and so forth. Other data and techniques may also or instead be used to track hormonal cycles. For example, cycle tracking may include a comparison of a user's RHR or HRV over time to the population model, or a cohort-specific or individual model for the user in order to infer a current day within the menstrual cycle. Other data and techniques, such as the ensemble techniques for detecting the phase within a hormonal cycle as described in U.S. Pat. Pub. No. 2024/0074709, or the analytical techniques for predicting a delivery date as described in U.S. Pat. Pub. No. 2024/0065616, may also or instead be used to monitor hormonal cycles. Each of the foregoing is incorporated herein by reference in its entirety. These techniques may generally be used in combination with one or more cardiovascular amplitude metrics described herein to support improved recommendations, observations, and other coaching and general feedback.

In general, coaching may be based on data such as but not limited to health metrics, menstrual cycle data, and/or the other data and metrics described herein, as well as user journaling, survey data, health provider data, and any other sources of data shared by a user and relevant to. Coaching may be based on an evaluation of, or conclusions drawn from, information for an individual and may include presenting conclusions or other information. In one aspect, coaching based on hormonal cycles may address macroscopic or lifestyle issues. For example, coaching may be directed to improving BMI, improving sleep quality, increasing movement, increasing exercise, reducing or otherwise managing stress levels, and so forth, with specific inferences and recommendations based on a user's journaled history, physiological data, activity detection, stress measurements, sleep activity, and so forth.

In another aspect, coaching based on hormonal cycles may address fertility. The cardiovascular amplitude metric may serve as a proxy for fertility. In one aspect, fertility or hormone cycle regularity may be increased by maintaining a healthy weight, and coaching may include information on how an individual may reach or maintain such a healthy weight. In another aspect, coaching may include suggestions on how to maintain a healthy diet and may explain the effects of diet on fertility and hormonal cycle. In another aspect, coaching may include information on how stress can affect fertility and may include suggestions on how to manage stress levels, e.g., based on an analysis of a user's stress history. Stress may be managed with activities such as exercise, meditation, yoga, and other relaxation exercises. Coaching may also or instead include suggestions for behavioral therapy, which may help an individual manage stress or may help an individual target behaviors and habits that may affect fertility or hormonal cycles. Coaching may include suggesting more sleep as a method for improving fertility, hormonal levels, or cycle regularity. Coaching may also include other lifestyle change suggestions, such as stopping smoking or limiting alcohol consumption and caffeine. Coaching may also include providing information about the most fertile days for conception, the likelihood of conception (which may be related to a high cardiovascular amplitude for a user), and a current day within the menstrual cycle relative to those most fertile days. In another aspect, coaching may include recommendations for nutrition and supplements.

Coaching may also or instead include other types of recommendations or feedback concerning other hormonal therapies and the like. In general, hormonal therapy can be used to regulate hormonal cycles and manage various symptoms related to hormonal imbalances. Because a metric such as cardiovascular amplitude can provide a proxy for user hormone levels, the cardiovascular amplitude metric may be used to inform a user's current or prospective hormone therapies. For example, hormonal therapies may be used to address menopause, premenstrual syndrome (PMS), or polycystic ovary syndrome (PCOS). In one aspect, Hormone Replacement Therapy (HRT) may be used for menopausal women to replace hormones (estrogen, progesterone) that the body no longer makes after menopause. In another aspect, birth control pills may be used in younger women to regulate menstrual cycles, manage PMS, or treat conditions like PCOS. Other medications may also or instead be used to manage hormonal conditions or imbalances, such as using anti-androgens or insulin sensitizers to treat PCOS. These and other treatments may be managed based on, e.g., information about an individual's hormonal cycle as derived, e.g., using a cardiovascular amplitude metric based on the techniques described herein.

More generally, coaching may include recommendations for hormone supplements, either generally, or specifically dosed for a user to adjust the user's cycle amplitudes so that they match healthy patterns, encourage fertility, address imbalances, and so forth. Using the techniques described herein, a current hormonal cycle may be monitored, and coaching may be provided in the form of recommendations for the amount and timing of hormones or other therapies synchronized with the measured hormonal cycle. Coaching may also include evaluating the effect of previous recommendations or drug therapies on an individual. In one aspect, coaching may specifically include a recommendation to consult a healthcare provider, e.g., where the user's physiological response to current treatments and/or other activities or behaviors does not match expected responses. In another aspect, coaching may include a recommendation for additional testing to confirm a suspicion for hormonal imbalances or irregularities that have been detected based on the cardiovascular amplitude metric. Thus, in one aspect, the methods and systems described herein may provide a continuous, non-invasive screener for hormonal issues that merit clinical investigation. When detected, a user may be proactively alerted or referred to a clinician for any suitable follow up tests or interventions.

In another aspect, the cardiovascular amplitude metric may be used to support hormone and fertility tracking. General hormonal health can impact the likelihood of conception, and a cardiovascular amplitude metric can be used in combination with other data to track month-to-month changes in general hormonal health, e.g., by building one or more ML models using certain features such as one or more of: deviations from historical patterns in RHR, HRV, temperature, respiratory rate; cardiovascular amplitude, cycles and variations from cardiovascular amplitude baseline amplitudes; amplitude of skin temperature and/or respiratory rate and variations from baseline amplitudes; chronic anovulation, which can be related to female infertility (no egg=no pregnancy); and/or other features such as length of cycle (e.g., longer/shorter than normal), variability of cycle (e.g., variations, in length or other features, month-to-month greater than normal), age, and menopause.

Cardiovascular amplitude metrics may also provide useful indicators for levels of hormones such as estrogen and progesterone. Thus, for example, a longitudinal decline in cardiovascular amplitude (e.g., a user goes from 4 bpm to 1 bpm) or a low amplitude absolute value (e.g., always at 0.5 bpm) may indicate that a user's hormones are not fluctuating as anticipated. Conversely, larger and more regular (e.g., less noisy day to day) changes in the underlying cardiovascular metric may yield a higher cardiovascular amplitude that indicates better hormonal health and a higher likelihood of conception.

In another aspect, cardiovascular amplitude metrics may support detection and management of menopause with machine learning techniques. For example, one or more ML models may be trained to identify the onset of menopause based on patterns in one or more cardiovascular metrics. Traditionally, menopause is not defined until one year without menstruation. However, the onset of menopause is a gradual process with health impact prior to this one-year marker. Therefore, it can be advantageous to identify perimenopause and likely menopause prior to the traditional point of diagnosis. To this end, an ML model can be trained on cardiovascular amplitude data (which will decline with decreasing changes in hormone levels that accompany menopause), along with one or more of the following features: deviations from historical patterns in RHR, HRV, temperature, and/or RR cycles; amplitude of skin temperature and/or respiratory rate and variations from baseline amplitudes; chronic anovulation, which can be related to female infertility (no egg=no pregnancy); and/or other features such as length of cycle (e.g., longer/shorter than normal), variability of cycle (e.g., variations, in length or other features, month-to-month greater than normal), and age.

In another aspect, a cardiovascular amplitude metric may be used to detect and manage a transition through postpartum to a substantially regular menstruation cycle. More generally, the detection of any of the above events based on a cardiovascular amplitude metric can be used to coach a user to obtain improved hormonal health under various conditions. For example, a cardiovascular amplitude metric for a user may support coaching and improved outcomes around fertility, menopause perimenopause, postpartum, and any other context where hormonal imbalance or irregularity imposes hormonal health issues for the user.

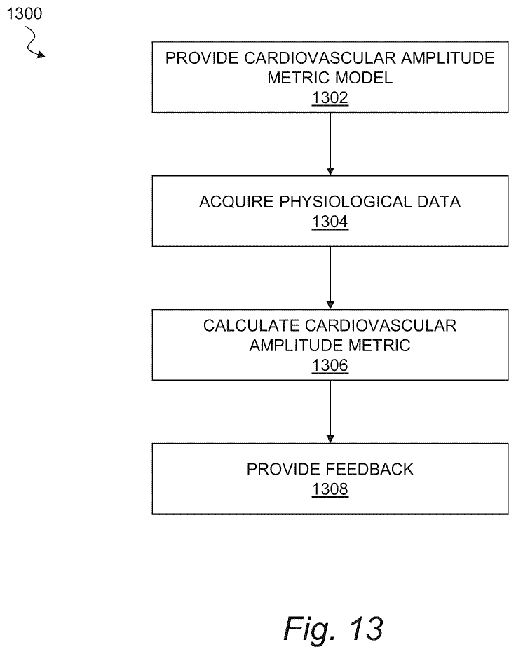

FIG. 13 shows a method for calculating and using a cardiovascular amplitude metric. In general, the cardiovascular amplitude metric described herein can provide a high-quality signal for the magnitude of changes in the amplitude of hormone-influenced physiological signals over the course of a menstrual cycle, thus providing a useful proxy for variations in hormone levels. This may be used as an independent metric for hormonal health or may be used to inform other hormone-related metrics and/or support coaching related to fertility, menopause, and other hormonal health or general health and wellness issues. In general, the model for evaluating a cardiovascular amplitude metric may be stored in a memory of any of the devices described herein for use in calculating the cardiovascular amplitude metric as needed.

As shown in step 1302, the method 1300 may include providing a model for a cardiovascular amplitude metric. In general, the model may be any of the models described herein that characterizes timewise changes in a cardiac metric of a user during a model hormonal cycle. The model hormonal cycle may, for example, be created using physiological measurements of a typical (e.g., mathematically averaged or otherwise aggregated) hormonal cycle for a particular user, a demographic cohort, or a population. This may include any of the physiological measurements, and any of the models and model hormonal cycles described herein. For example, the model hormonal cycle may include averaged resting heart rate or heart rate variability measurements for a user captured over the course of a menstrual cycle using any suitably consistent measurement methodology such as any of those described herein. This model may be used to identify typical turning points such as maxima and minima and characterize when these turning points occur during the menstrual cycle. The chronological location of these turning points (e.g., specific days within the menstrual cycle) may be used to construct sampling intervals or windows that are then used to acquire new user data when calculating the cardiovascular amplitude metric for a current user.

Thus, the model may provide or specify an interval for sampling a physiological metric for a user. For example, in one aspect, the model may be based on an offset between (a) a follicular mean over a first interval of at least three sequential daily measurements of resting heart rate or heart rate variability that includes day five of the model hormonal cycle and (b) a luteal mean over a second interval of at least three sequential daily measurements of a resting heart rate or a heart rate variability that includes day twenty five of the model hormonal cycle. More generally, the model may characterize timewise changes in a cardiac metric of a user during a model hormonal cycle, where the model is based on an offset between a first mean of a first interval of measurements around an expected follicular turning point of the cardiac metric for the user and a second mean of a second interval of measurements around an expected luteal turning point of the cardiac metric for the user.

As noted above, a variety of models may be used to calculate a cardiovascular amplitude metric, e.g., based on a cardiac metric or other physiological metric as described herein. For example, in one aspect, the model for the cardiovascular amplitude metric may characterize timewise changes in a cardiac metric for a user based on an offset between a follicular mean of the cardiac metric and a luteal mean of the cardiac metric, e.g., using any of the techniques described herein. The follicular mean may, for example, include a first mean of a first sequence of cardiac measurements over a first interval around an expected follicular turning point of the cardiac metric for the user, and the luteal mean may include a second mean of a second sequence of cardiac measurements around an expected luteal turning point of the cardiac metric for the user.

By way of more specific examples, in one aspect, the cardiac metric includes a resting heart rate; the expected follicular turning point of the cardiac metric occurs on day five of the hormonal cycle; the expected follicular turning point is a minimum of the resting heart rate; the first interval is at least seven days; the expected luteal turning point of the cardiac metric occurs on day twenty five of the hormonal cycle; the expected luteal turning point is a maximum of the resting heart rate; and the second interval is at least seven days. The first interval may instead be five days, four days, three or more days, or any number of days suitable for acquiring a consistent measure of the cardiac metric at the luteal turning point. In one aspect, an interval of seven days mitigates weekend effects where significant transient changes in lifestyle can affect cardiac metrics in a manner uncorrelated to hormonal cycles. In another aspect, the first interval may begin at the first or second day of the menstrual cycle and/or the second interval may end at the last or second to last day of the menstrual cycle. In another specific example, the cardiac metric includes a heart rate variability; the expected follicular turning point of the cardiac metric occurs on day five of the hormonal cycle; the expected follicular turning point is a maximum of the heart rate variability; and the first interval is at least three days. In this example, the cardiac metric may include a heart rate variability; the expected luteal turning point of the cardiac metric may occur on day twenty five of the hormonal cycle; the expected luteal turning point may be a minimum of the heart rate variability; and the interval may be at least three days. In each of the preceding examples, the location and duration of the first and/or second interval may vary as described herein.

As shown in step 1304, the method 1300 may include acquiring physiological data. This may, for example, include acquiring physiological data from any of the monitors described herein, such as a wearable photoplethysmography monitor or other wearable physiological monitor or the like configured to acquire data such as cardiac data, heart rate data, or other data. In one aspect, the monitor may include a wearable physiological monitor. The monitor may also or instead advantageously include a wearable continuous physiological monitor suitable for acquiring, e.g., continuous data over the course of a menstrual cycle, and more particularly, over sleep intervals or other intervals throughout the hormonal cycle during which consistent measurements can be taken.

As shown in step 1306, the method 1300 may include calculating the cardiovascular amplitude metric of the user for a particular user hormonal cycle with the model for the cardiovascular amplitude metric. In general, calculating the cardiovascular amplitude metric may be performed locally on a personal computing device for a user (e.g., a laptop, smart phone, desktop, etc.) or on a remote server where the user can access the information through a website or other network-based user interface. Calculating the cardiovascular amplitude may generally include acquiring a number of sequential measurements of the corresponding cardiac metric, e.g., over any of the intervals described herein around a follicular and a luteal turning point in the cardiac metric. A mean, median, or other representative value for each of these sequential cardiac metrics may then be calculated, and the difference between the representative value at each turning point can then be evaluated to determine the cardiovascular amplitude metric representative of the peak-to-peak variation in the underlying cardiac metric over the course of a menstrual cycle.

As shown in step 1308, the method 1300 may include providing feedback to the user based on the cardiovascular amplitude metric. The feedback may include any combination of the feedback and coaching described herein. For example, providing feedback to the user may include displaying the cardiovascular amplitude metric to the user, e.g., in a user interface of the physiological monitoring device, a personal computing device associated with the user, or any of the other computing devices described herein. In another aspect, providing feedback to the user may include estimating a phase of the user in a current hormonal cycle (e.g., using any of the techniques described herein); determining a confidence level for the phase based on the cardiovascular amplitude metric; and reporting the confidence level for the phase to the user. In general, the greater the cardiovascular amplitude of the physiological signal over the menstrual cycle, the more accurate the cycle timing can be, and the greater the resulting confidence in the resulting phase estimate. This may be represented, e.g., as a qualitative measure of confidence (e.g., high, moderate, low), or as a quantitative measure of confidence (e.g., on a scale of 0-10).

In another aspect, providing feedback to the user may include providing fertility coaching such as any of the fertility coaching described herein. For example, providing fertility coaching may include estimating a phase of the user in a current hormonal cycle; calculating an ovulation time of the user based on the phase; and reporting a confidence level in the ovulation time to the user based on the cardiovascular amplitude metric. In another aspect, providing fertility coaching may include reporting a likelihood of conception to the user. While the user-specific likelihood may be difficult or impossible to accurately predict, and may depend on numerous factors outside the scope of physiological monitoring, a relative likelihood of conception for the user can readily be established based on the general relationship between high cardiovascular amplitude and hormonal health, and may be reported to the user in suitably broad terms and with suitable caveats. Providing fertility coaching may also or instead include providing suggestions for one or more behavioral modifications to increase the cardiovascular amplitude metric.

In another aspect, fertility coaching may include suggestions on timing for related activities. For example, providing fertility coaching may include determining a phase of the user based on data from the physiological monitor; storing one or more ovulation observations by the user in a memory; predicting an ovulation time for the user based on a combination of the phase of the user and the one or more ovulation observations; and providing a fertility coaching recommendation to the user including the ovulation time, a likelihood to conceive based on the cardiovascular amplitude metric, and an accuracy of the ovulation time based on the cardiovascular amplitude metric.

In another aspect, the feedback may include an indicator to a user of a possible onset of perimenopause or menopause. This may be detected, e.g., based on a decreasing trend in the cardiovascular amplitude over a number of hormonal cycles. The indicator may be provided, e.g., as an email or text alert, or within a user interface of a personal computing device for the user. In another aspect, the feedback may include a referral to a clinician when warranted by changes (or lack thereof) in the cardiovascular amplitude metric, either alone or in combination with other data from a physiological monitor and/or information provided by the user concerning symptoms, wellness goals, and so forth. Feedback may also or instead include any feedback based on inferences from machine learning models trained with cardiovascular amplitude data and any other suitable data, along with appropriate labeling, to identify, e.g., menopause, perimenopause, changes in fertility, postpartum timing, and so forth.

According to the foregoing, in one aspect there is disclosed herein a system including a physiological monitor, a memory, and a processor. The physiological monitor may include a wearable physiological monitor configured to acquire cardiac data from a user. The memory may store a model for a cardiovascular amplitude metric that characterizes timewise changes in a cardiac metric of a user during a model hormonal cycle, where the model is based on an offset between a first mean of a first interval of measurements around an expected follicular turning point of the cardiac metric for the user and a second mean of a second interval of measurements around an expected luteal turning point of the cardiac metric for the user. The processor may be configured by computer executable code to receive data from the wearable physiological monitor over a hormonal cycle for the user, calculate the cardiovascular amplitude metric for the hormonal cycle, and generate feedback to the user based on the cardiovascular amplitude metric, such as by displaying the cardiovascular amplitude or related information in a user interface associated with, e.g., any of the computing devices described herein.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in a computer program product such as computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

1. A computer program product comprising computer executable code embodied in a non-transitory computer readable medium that, when executing on one or more computing devices, causes the one or more computing devices to perform the steps of:
providing a model for a cardiovascular amplitude metric that characterizes timewise changes in a cardiac metric of a user during a model hormonal cycle based on an offset between (a) a follicular mean over a first interval of at least three sequential daily measurements of resting heart rate or heart rate variability that includes day five of the model hormonal cycle and (b) a luteal mean over a second interval of at least three sequential daily measurements of a resting heart rate or a heart rate variability that includes day twenty five of the model hormonal cycle;
acquiring heart rate data for the user from a wearable photoplethysmography monitor during a user hormonal cycle;
calculating the cardiovascular amplitude metric of the user for the user hormonal cycle with the model for the cardiovascular amplitude metric; and
providing feedback to the user based on the cardiovascular amplitude metric.

2. The computer program product of claim 1, wherein providing feedback to the user includes displaying the cardiovascular amplitude metric to the user.

3. The computer program product of claim 1, wherein providing feedback to the user includes:
estimating a phase of the user in a current hormonal cycle;
determining a confidence level for the phase based on the cardiovascular amplitude metric; and
reporting the confidence level for the phase to the user.

4. The computer program product of claim 1, wherein providing feedback to the user includes providing fertility coaching.

5. The computer program product of claim 4, wherein providing fertility coaching includes:
estimating a phase of the user in a current hormonal cycle;
calculating an ovulation time of the user based on the phase; and
reporting a confidence level in the ovulation time to the user based on the cardiovascular amplitude metric.

6. The computer program product of claim 4, wherein providing fertility coaching includes reporting a likelihood of conception to the user.

7. The computer program product of claim 4, wherein providing fertility coaching includes providing suggestions for one or more behavioral modifications to increase the cardiovascular amplitude metric.

8. The computer program product of claim 4, wherein providing fertility coaching includes:
determining a phase of the user based on data from the wearable photoplethysmography monitor;
storing one or more ovulation observations by the user in a memory;
predicting an ovulation time for the user based on a combination of the phase of the user and the one or more ovulation observations; and
providing a fertility coaching recommendation to the user including the ovulation time, a likelihood to conceive based on the cardiovascular amplitude metric, and an accuracy of the ovulation time based on the cardiovascular amplitude metric.

9. The computer program product of claim 1, wherein providing feedback includes providing an indicator to the user of a possible onset of perimenopause or menopause.

10. A method comprising:
providing a model for a cardiovascular amplitude metric that characterizes timewise changes in a cardiac metric for a user based on an offset between a follicular mean of the cardiac metric and a luteal mean of the cardiac metric;
acquiring physiological data for the user from a wearable monitor during a hormonal cycle, wherein the physiological data includes heart rate data for the user;
calculating the cardiovascular amplitude metric of the user with the model for the hormonal cycle based on the physiological data; and
providing feedback to the user based on the cardiovascular amplitude metric.

11. The method of claim 10, wherein providing feedback to the user includes displaying the cardiovascular amplitude metric to the user.

12. The method of claim 10, wherein providing feedback to the user includes providing fertility coaching.

13. The method of claim 12, wherein providing fertility coaching includes:
estimating a phase of the user in a current hormonal cycle;
calculating an ovulation time of the user based on the phase; and
reporting a confidence level in the ovulation time to the user based on the cardiovascular amplitude metric.

14. The method of claim 12, wherein providing fertility coaching includes reporting a likelihood of conception to the user.

15. The method of claim 12, wherein providing fertility coaching includes:
determining a phase of the user based on data from the wearable monitor;
storing one or more ovulation observations by the user in a memory;
predicting an ovulation time for a user based on a combination of the phase of the user and the one or more ovulation observations; and
providing a fertility coaching recommendation to the user including the ovulation time, a likelihood to conceive based on the cardiovascular amplitude metric, and an accuracy of the ovulation time based on the cardiovascular amplitude metric.

16. The method of claim 10, wherein the follicular mean includes a first mean of a first sequence of cardiac measurements over a first interval around an expected follicular turning point of the cardiac metric for the user, and wherein the luteal mean includes a second mean of a second sequence of cardiac measurements over a second interval around an expected luteal turning point of the cardiac metric for the user.

17. The method of claim 16, wherein:
the cardiac metric includes a resting heart rate;
the expected follicular turning point of the cardiac metric occurs on day five of the hormonal cycle;
the expected follicular turning point is a minimum of the resting heart rate;
the first interval is at least seven days;
the expected luteal turning point of the cardiac metric occurs on day twenty five of the hormonal cycle;
the expected luteal turning point is a maximum of the resting heart rate; and
the second interval is at least seven days.

18. The method of claim 16, wherein:
the cardiac metric includes a heart rate variability;
the expected follicular turning point of the cardiac metric occurs on day five of the hormonal cycle;
the expected follicular turning point is a maximum of the heart rate variability; and
the first interval is at least three days.

19. The method of claim 16, wherein:
the cardiac metric includes a heart rate variability;
the expected luteal turning point of the cardiac metric occurs on day twenty five of the hormonal cycle;
the expected luteal turning point is a minimum of the heart rate variability; and
the second interval is at least three days.

20. A system comprising:
a wearable physiological monitor configured to acquire cardiac data from a user;
a memory storing a model for a cardiovascular amplitude metric that characterizes timewise changes in a cardiac metric of a user during a model hormonal cycle, wherein the model is based on an offset between a first mean of a first interval of measurements around an expected follicular turning point of the cardiac metric for the user and a second mean of a second interval of measurements around an expected luteal turning point of the cardiac metric for the user; and
a processor configured by computer executable code to receive data from the wearable physiological monitor over a hormonal cycle for the user, calculate the cardiovascular amplitude metric for the hormonal cycle, and generate feedback to the user based on the cardiovascular amplitude metric.

21-25. (canceled)
